(19) United States
(12) Patent Application Publication
(10) Pub. No.: US 2010/0069803 A1
(43) Pub. Date: Mar. 18, 2010

(54) AIR REACTING/SETTING CAST CONTAINED WITHIN AN AIR PERMEABLE ENCLOSURE OR FLEXIBLE/OPENABILE BAG IN ORDER TO PROVIDE A FLEXIBLY APPLIED AND HARDENED TEMPORARY CAST

(75) Inventor: Miguel A. Linares, Bloomingfield Hills, MI (US)

Correspondence Address: GIFFORD, KRASS, SPRINKLE,ANDERSON & CITKOWSKI, P.C, PO BOX 7021, TROY, MI, 48007-7021, US

(73) Assignee: LINARES MEDICAL DEVICES, LLC, Auburn Hills, MI (US)

(21) Appl. No.: 12/560,493

(22) Filed: Sep. 16, 2009

Related U.S. Application Data
(60) Provisional application No. 61/097,340, filed on Sep. 16, 2008.

(51) Int. Cl. A61F 5/00

(52) U.S. Cl. 602/6

(57) ABSTRACT

A temporary cast consisting of a material exhibiting a specified length, width and thickness, such as an elongated and wound roll material impregnated with a fluid hardenable material. The roll material is retained (in an unused state) encased within an air impermeable container or, in a further application, can exhibit a surface applied and rupture-able foil material to prevent the fluid entrained within the cast material to prematurely harden. The fluid hardening component associated with said material is such that upon exposure to ambient air, is capable of being wound or otherwise formed about the injured location and prior to it being caused to set and harden.

### CROSS REFERENCE TO RELATED APPLICATIONS

The present application claims the priority of U.S. provisional application Ser. No. 61/097,340 filed Sep. 16, 2008.

### FIELD OF THE INVENTION

The present invention relates generally to an air reacting/settable cast. More specifically, the present invention discloses an air reacting/setting cast, such as in the form of a wrap-around material which is contained within an air-tight enclosure (including such as a box-like container which supports a wound roll of elongated material as well as a sealed bag enclosure within which the material is contained as a wrap-around fabric). Upon the material being removed from the box (or bag enclosure) and pre-positioned over an injury location, exposure to the ambient air causes the cast material to chemically react and harden, such as in order to provide temporary protection to the injured limb/location. The splint assembly is advantageously used by first responders or other emergence medical personal, for the purpose of immobilizing a wounded limb, and until such time as appropriate professional medical personnel can substitute the splint with a limb cast or the like.

### DESCRIPTION OF THE PRIOR ART

The prior art is documented with various types of temporary cast and splint assemblies, such as which are employed by emergency medical personnel and other first responders. An objective of such cast and splint assemblies is to immobilize fractured and sprained bones associated with various limbs, such as during transport of the wounded individual to a hospital or other medical care facility.

Disadvantages of existing splint assemblies include both their relative weight and bulkiness. This is particular an issue in instances where space is limited in EMS vehicles.

### SUMMARY OF THE PRESENT INVENTION

The present invention discloses a temporary cast which consists of a material exhibiting a specified length, width and thickness, such as an elongated and wound roll material impregnated with a fluid hardenable material. The roll material, in one application, can be retained (in an unused state) encased within an air impermeable container or, in a further application, can exhibit a surface applied foil material or the like to prevent the fluid entrained material to prematurely harden. Upon removal from the enclosure, and/or upon rupturing an associated foil seal in order to expose the fluid entrained cast or fabric material to ambient air, the material is capable of being wound or otherwise formed about the injured location and prior to it being caused to harden.

Additional features include the material further formed as a foam layer with and an adhering plasticized layer and including a chemical additive which reacts with said material to promote hardening. In one particular application, an aperture is defined in a surface of the box enclosure and such that, upon passing the strip therethrough in a frictional and abrading edge contacting fashion, causes a foil surface layer to rupture and to promote chemical reaction and hardening of the fluidic hardening component with the ambient air.

In a further application, the fluid impregnated material can also include a wrap around planar article exhibiting a more generally rectangular shaped and which is contained within a bag enclosure, such as further including a hermetically sealed or rupture-able opening. Upon removing the material from the bag, an associated foil seal is ruptured (or in the absence of the seal) the introduction of ambient air is caused to progressively harden the sheet following it being formed about a desired location.

### BRIEF DESCRIPTION OF THE DRAWINGS

Reference will now be made to the attached drawings, when read in combination with the following detailed description, wherein like reference numerals refer to like parts throughout the several views, and in which:

FIG. 1 is an illustration of the cast material provided as a wound roll within a generally box-shaped container according to a first preferred embodiment of the present invention;

FIG. 2 is an enlarged partial illustration of section 2-2 of the cast material shown in FIG. 1 and further illustrating it's foam and hard plastic layers;

FIG. 3 is a further partial illustration of an apertured location in the box which is configured to frictionally abrade the cast material as it is drawn through the opening, and in order to trigger the chemical reaction to harden the cast material upon it being applied to the wearer;

FIG. 4 is an environmental illustration of the cast material of FIG. 1 applied to the limb of a user;

FIG. 5 is an illustration of a wrap-around cast design pattern similar to that illustrated in FIG. 1;

FIG. 6 is an illustration of a sealed bag enclosure within which the material is contained as a wrap-around fabric according to a further preferred embodiment of the present invention;

FIG. 7 is an enlarged partial illustration of the cast material shown in FIG. 6;

FIG. 8 is an illustration of a cast design pattern according to the preferred embodiment of FIG. 6;

FIG. 9 is an illustration of the fluid impregnated material in the form of a wrap around planar article exhibiting a more generally rectangular shaped and which is contained within a bag enclosure, such as further including a hermetically sealed or rupture-able opening; and

FIG. 10 is a succeeding illustration of the variant of FIG. 9 and which, upon removing the sheet material from the bag, an associated foil seal is ruptured (or in the absence of the seal) the introduction of ambient air is caused to progressively harden the sheet following it being formed about a desired location.

### DETAILED DESCRIPTION OF THE PREFERRED EMBODIMENTS

The present invention discloses an air reacting/setting cast, such as in the form of a wrap-around material which is contained within an air-tight enclosure (including such as a box-like container which supports a wound roll of elongated material as well as a sealed bag enclosure within which the material is contained as a wrap-around fabric). As will be described, and upon the material being removed from the box (or bag enclosure) and pre-positioned over an injury location, exposure to the ambient air causes the cast material to chemically react and harden, such as in order to provide temporary protection to the injured limb/location. The splint assembly is advantageously used by first responders or other emergence medical personal, for the purpose of immobilizing a wounded limb, and until such time as appropriate professional medical personnel can substitute the splint with a limb cast or the like.

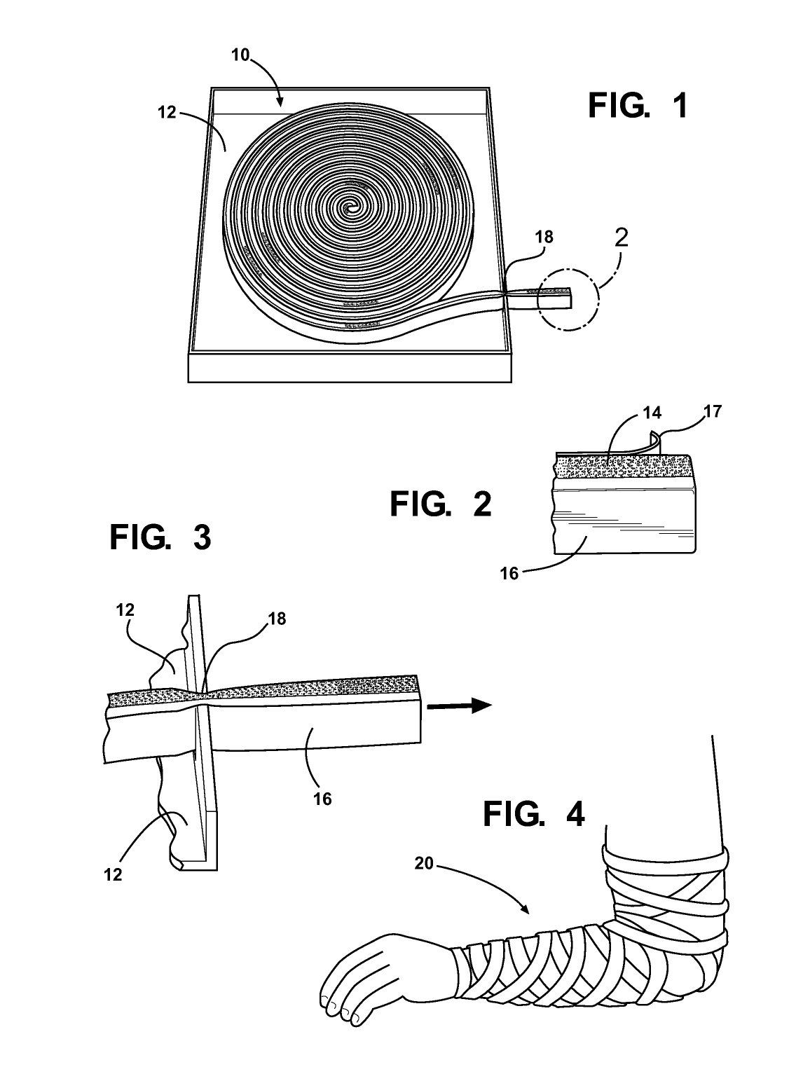

FIG. 1 is an illustration of 10 of the cast material provided as a wound roll within a generally box-shaped container 12 (illustrated in open cutaway and without a corresponding lid or top in place) according to a first preferred embodiment of the present invention. In a preferred embodiment, the wound roll is provided according to any desired configuration (including such as a generally ribbon, mat or other rectangular shape) and is maintained in a closed (optionally airtight sealed) container. It is also understood that the container 12 can exhibit either a rigid or soft construction, as well as optionally being airtight.

Referring further to FIG. 2, an enlarged partial illustration is shown of the cast material in FIG. 1, and which illustrates a first foam (or optionally fabric) layer 14 and a second (hard) plastic over-layer 16. The foam layer 14 operates as a contact surface and can optionally also include a fabric (including gauze) or other sanitary component within which is entrained a suitable fluid entrained and hardenable material. The plastic layer 16 (such as a sanitary plastic) functions as a semi-rigid backing layer and which, in combination with the chemical reacting/hardening properties to be described herein, operates to create a rigid cast material which is capable of being quickly applied to such as a sprained or fractured limb, rib or other appropriate location. It is understood that the provision of a backing or supporting layer, such as again shown at 16, is optional and that, in addition to forming an applying portion of the cast in one embodiment, can also be provided as a form supporting and disposable backing sheet in a further variant, and by which only the intermediately located foam or fabric layer 14 is employed and hardened upon rupturing or peel away removal of the hermetical seal/foil layer.

Also shown in FIG. 2 is an optional foil layer 17, such as which is capable of being secured to an exposed surface of the fluid impregnated foam or fabric layer 14. The foil layer 17 operates to prevent premature hardening of the fluid impregnated material and is understood in one embodiment to cover the entire surface area of the fluid impregnated foam and/or fabric material 14, with the edges of the foil layer 17 contacting the plastic or like layer 16.

As further shown in each of FIG. 1 and the further partial illustration of FIG. 3, an apertured location is illustrated by inner facing walls 18 in the box 12, this being configured and dimensioned to allow for unwinding withdrawal of the wound roll (such as further in order to frictionally abrade the thin surface covering foil 17) as the cast material (again including layers 14 and 16) is progressively drawn through the opening 18 (see also directional arrows referenced in FIG. 3). While the invention is not limited to any specific ranges of thicknesses or dimensions, one non-limiting range can include such as ¼″ to ½″ overall thickness of the foam/fabric layer, with a plastic backing layer providing a similar dimension and an optional sealing foil material a further minor dimension, such as equal or less than 1/16″. As will be further described with reference to the several succeeding embodiments, the cast forming material can include any length (such as incorporated into a roll) and with a thickness ranging from the relatively narrow (½″ to several inches) up to a planar sheet roll of 1′ or greater, this based upon the desired application.

Consistent with the above description, either or both of the layers 14 and 16 of material can incorporate a chemical additive, such as which is triggered by virtue of the frictional squeezing/abrading contact established between the layers and the side walls of the aperture (such as further contemplating the incorporation of a suitable knife edge or the like associated with an inner wall 18 of the aperture and to facilitate rupturing of the foil seal 17 or otherwise triggering of the entrained chemical component associated with the fluid impregnated layer). This is again in order to trigger the chemically reactive component to react with the ambient air to harden the cast material in a relatively short period of time following it being pre-wrapped around (or pre-positioned upon) the wearer. It is further envisioned that the foil layer can be dispensed with and the chemical makeup of the impregnated fluid material modified such that it will initiate a hardening reaction of the foam layer 14, upon being pinched or otherwise abraded in the manner shown in FIG. 3.

The hardening material can further include any of a number of different chemical additives, such as which are known in the relevant art to chemically react with the given foam and/or plasticized material layers and in order to quickly set and dry in the desired wound position about the wearer (see FIG. 4). The chemical additive can be calibrated to exhibit a drying time of less than 10 minutes, although other faster drying compositions are possible and which can be substituted in given applications.

It is also understood and envisioned that the foam and hardened plastic layers of the elongated strip material shown in FIG. 1 can be reconfigured as a single layer of material, such exhibiting a composite mixture of elements and further including the desired chemical additive/reactant. Other variants also contemplate the provision of a peel-away surface layer of material such as which is positioned in similar fashion to the rupturable foil layer 17 and which is removed either prior or following to being applied to a skin surface of the desired area in order to initiate air-contact drying/setting of the cast material, such as further once wound in place in its desired application.

FIG. 4 is an environmental illustration of the cast material of FIG. 1, generally referenced at 20, and which is applied to the limb of a user. The illustration shown represents one of many possible variations of the cast design which can be applied to the user and without departing from the scope of the invention.

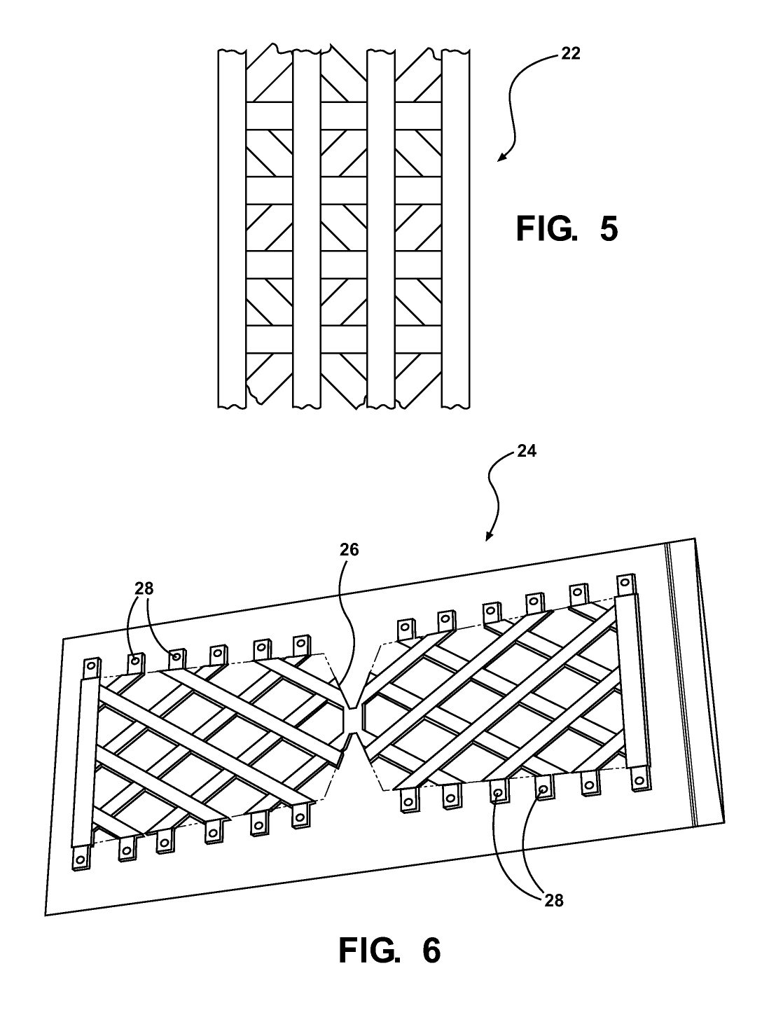

Referring also to FIG. 5, an illustration is shown at 22 of a wrap-around cast design pattern, similar to that illustrated in FIG. 1, and representing one additional possible variation for binding and securing the settable cast material about a limb/body location of a user and which contemplates the provision of a honeycomb or grid-like appearance. It is envisioned that a suitable foil seal template can be provided for this variant, however it is also understood that a flat backing sheet (not shown) can also be provided and which is nominally in contact with a permeable two dimensional exposed face of the tourniquet style design of FIG. 5 and which can also include a fluid impregnated and harden-able consistency.

Referring now to FIG. 6, an illustration is generally shown at 24 of a sealed bag enclosure, and within which a related version of the cast material 26 is contained as a wrap-around fabric according to a further preferred embodiment of the present invention. The variant 24 differs from that previously shown at 10, primarily in that the material 26 can be folded or otherwise applied, and as opposed to wrapping in multiple passes about a limb or trunk location of the user.

The variant of FIG. 6 also differs in that the entrained chemical additive may not require abrading engagement with an aperture edge surface or the like, and in order to be activated to set/dry, such as again within a 5-10 minute period of time. As further shown in FIG. 6, a series of snaps or end tabs, see at 28, may be provided, and which facilitate the application/securing and subsequent hardening into the desired temporary cast configuration.

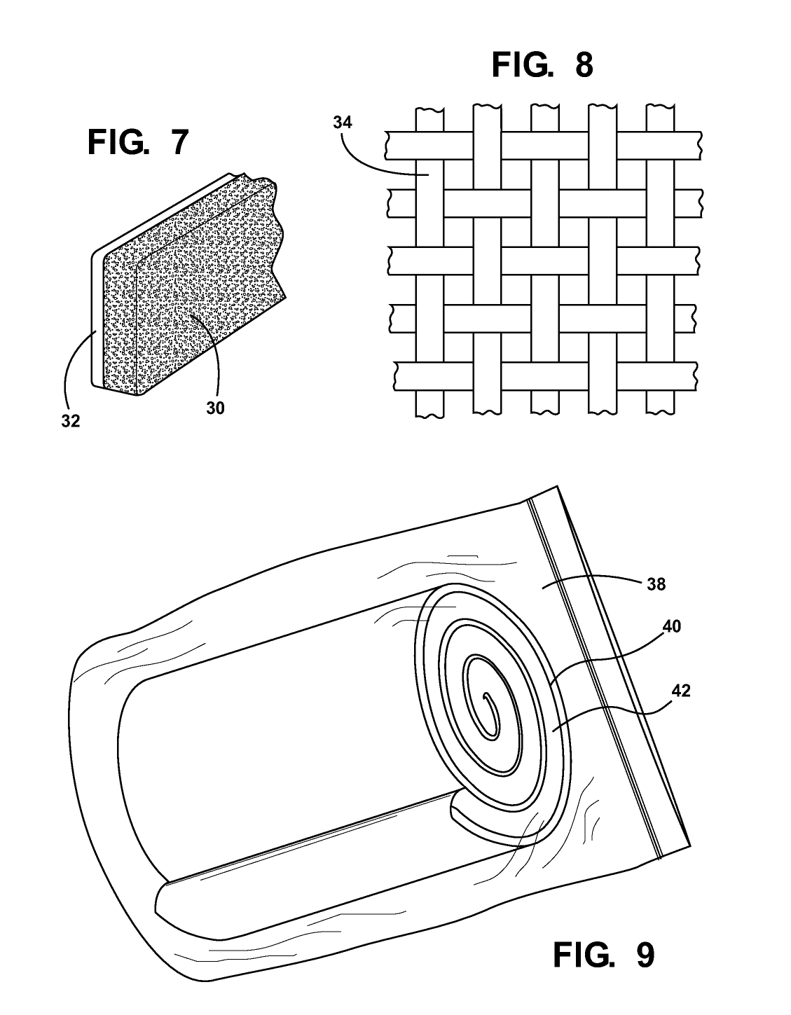

In application, the cast material 26, such as is provided as a fold-over article as shown, is removed from the sealed bag (or other suitable enclosure), following which it is wrapped or otherwise bound about a limb, torso or other suitable location of a wearer. As previously described, the temporary cast material is entrained or impregnated with a chemical additive which reacts with the material content of the cast (this again shown in reference to the enlarged partial illustration in FIG. 7 and including such as a first foam layer 30 and a second flexible plastic backing layer 32). Although not shown in this illustration, a suitable rupture-able foil or peel away layer (see as previously again shown at 17 in FIG. 2), can be provided in three dimensional covering fashion around the impregnated foam layer and in edge contacting fashion with the flexible plastic backing.

Unlike the winding applied variant 10 of FIG. 1, the chemical reacting/hardening component associated with the bag contained fold-over article 26 may be activated simply by removing the article from the bag and, upon applying to the desired location, allowing contact with ambient air (and not necessarily initiated by frictionally and/or resistively actuating a surface of the article through an abrading contact with a wall surface as is illustrated in FIG. 1). It is also again envisioned that a peel-away backing layer, or other reaction initiating process, can be incorporated into the material 26, such as which is intended to promote fast drying (e.g. 5-10 minutes in certain instances) of the applied article.

Referring to FIG. 8, an illustration 34 is shown of a further envisioned latticework pattern associated with any of the settable cast materials. In addition to each of the embodiments previously described, the temporary cast design can also be provided as a single layer containing a composite of both flexible and rigid material components. Alternately, the cast construction can exhibit multiple layers in which the hardening promoting chemical additive is entrained within some or all of the layers and which may further include the provision of a rupturable thin foil or peel away layer (and which can exhibit thicknesses such as ranging from 20 mil up to 1/16″ in one non-limiting variant).

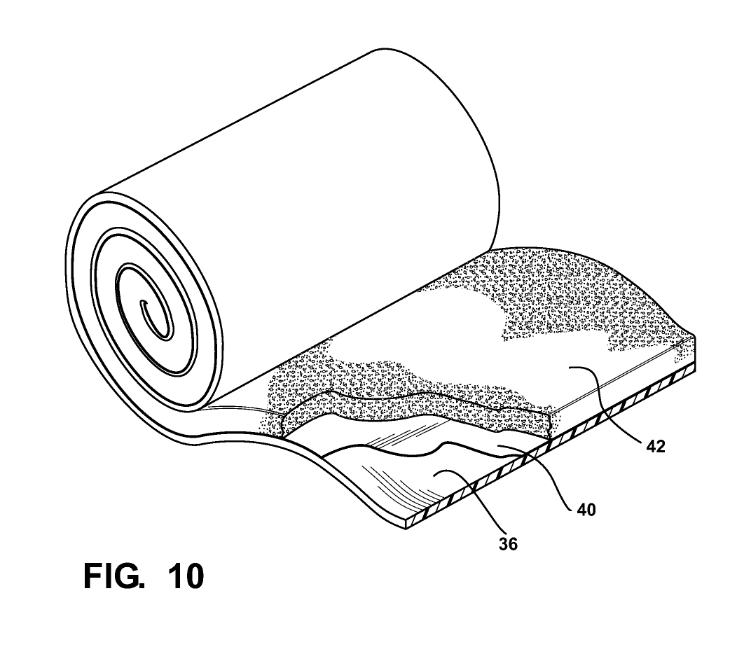

Referring finally to FIGS. 9 and 10, a further application contemplates a fluid impregnated material exhibited as an alternatively configured wrap around planar article, such having a generally rectangular shaped backing layer 36 and which is contained within a bag enclosure 38. The bag can include a hermetically sealed opening (or alternatively a foil-like rupture-able opening), or can include a resealable top edge consistent with known bag designs.

Upon removing the material from the bag, an associated foil seal 40 is applied over a fluid impregnated foam or fabric layer 42. In the illustrated embodiment, the foil seal 40 is shown in sandwiched fashion between the foam layer 42 and the flexible backing layer 36 and such that the backing layer can be disposed of at the point of use and the seal ruptured. Alternately, the foil layer 40 can be repositioned on the inner exposed surface of the three dimensional and fluid impregnated layer 42 and the flexible backing sheet 36 can form a permanent part of the eventual cast.

In the absence of a pre-sealed environment, the introduction of ambient air created by rupturing a previously hermetically sealed bag, thereby causing the sheet to progressively harden further such as following it being formed about a desired location. The layer 42 can include any admixture of fabric, gauze and/or polymeric components (formed into one layer or into multiple layers as previously described) and such that it can be provided as a unitary and fluid impregnated layer which is caused by the chemical setting of the fluid to harden into a desired and previously formed shape (see again exemplary environmental illustration of FIG. 4).

It is also envisioned that applications of the splint assembly can also be reconfigured to function as a long-term and enduring cast configuration. The variously sized splint subassemblies can again being carried by first response (e.g. EMS or paramedic) personnel, and which are quickly and effortlessly applied to secure and immobilize an individual's limb until such time as appropriate medical care can be applied.

Having described my invention, other and additional preferred embodiments will become apparent to those skilled in the art to which it pertains, and without deviating from the scope of the appended claims.

I claim:

1. A settable cast, comprising:
a flexible material exhibiting a length, width and thickness; and
a settable fluidic component entrained within said flexible material and which is chemically reacted in order to harden within a set time.

2. The cast as described in claim 1, further comprising at least one of a rupture-able foil or a peel away layer applied over said flexible material and initiating said chemical reaction.

3. The cast as described in claim 1, further comprising an enclosure within which said material is retained in a stored state.

4. The cast as described in claim 3, said enclosure having an aperture through which said flexible material is translated in abrading fashion in order to trigger said chemical setting reaction.

5. The cast as described in claim 1, said material further comprising a foam layer and an adhering flexible layer.

6. The cast as described in claim 1, said material further comprising an elongated and wound strip contained within an enclosure.

7. The cast as described in claim 1, said material further comprising a wrap around planar article contained within a bag enclosure.

8. A settable cast, comprising:
a flexible material having a length, width and thickness and which includes at least one layer impregnated with a chemically reacting and hardenable fluid component;
an enclosure for containing said flexible material; and
upon removal of said material from said enclosure and exposure to ambient air, said material is adapted to being applied to a use location and is caused to harden.

9. The invention as described in claim 8, said flexible material further comprising at least one of a foam or fabric layer.

10. The invention as described in claim 8, further comprising a second flexible backing layer applied to said flexible material.

11. The invention as described in claim 8, further comprising an air impermeable and rupture-able foil layer applied over said impregnated layer.

12. The invention as described in claim 7, said enclosure having a rigid construction and including an aperture through which said flexible material is removed.

13. The invention as described in claim 12, said aperture further comprising a narrowed edge profile associated with an inner wall of said aperture and through which said flexible material is withdrawn in at least one of a pinching and abrading contacting fashion.

14. The invention as described in claim 8, said flexible material further comprising at least one of a ribbon, roll or wound rectangular mat.

15. The invention as described in claim 8, further comprising an air impermeable peel-away applied over said impregnated layer.

16. The invention as described in claim 10, said flexible backing layer further comprising a sanitary plastic.

17. The invention as described in claim 16, said backing layer further comprising a disposable faun supporting layer.

18. A settable cast, comprising:
a flexible material including at least one of a foam or a fabric and having a length, width and thickness impregnated with a chemically reacting and hardenable fluid component;
an enclosure for containing said flexible material said having a rigid construction with an aperture lesser than said nominal thickness of said material and through which said flexible material is abradingly removed to trigger said chemical reaction; and
upon removal of said material from said enclosure, said material is applied to a use location and is caused to harden.

19. The invention as described in claim 18, said flexible material further comprising at least one of a ribbon, roll or wound rectangular mat.

20. The invention as described in claim 18, further comprising an air impermeable and rupture-able foil layer applied over said impregnated layer.
